(19) United States
(12) Patent Application Publication
(10) Pub. No.: US 2014/0228311 A1
(43) Pub. Date: Aug. 14, 2014

(54) CRYSTALLIZATION OF IDARUBICIN HYDROCHLORIDE

(71) Applicant: Tero Kunnari, Aschaffenburg (DE)

(72) Inventor: Tero Kunnari, Aschaffenburg (DE)

(73) Assignee: HERAEUS PRECIOUS METALS GMBH & CO. KG, Hanau (DE)

(21) Appl. No.: 14/345,685

(22) PCT Filed: Sep. 4, 2012
(86) PCT No.: PCT/EP2012/003691

§ 371 Date: Mar. 19, 2014

Related U.S. Application Data
(60) Provisional application No. 61/536,240, filed on Sep. 19, 2011.

(30) Foreign Application Priority Data
Sep. 19, 2011 (DE) 10 2011 113 652.9

(51) Int. Cl. C07H 15/252

(52) U.S. Cl. CPC C07H 15/252
USPC 514/34; 536/6.4

(57) ABSTRACT

A method is provided for production of crystalline idarubicin hydrochloride, the method including the steps of: (i) producing a mixture containing (a) idarubicin hydrochloride, (b) at least one alcohol selected from 1-butanol, 2-butanol, and 1-pentanol, and (c) water; and (ii) crystallizing idarubicin hydrochloride from this mixture. A crystalline idarubicin hydrochloride is also provided characterized by a powder x-ray diffraction pattern in which at least reflexes at diffraction angles occur in the following ranges (in 2Θ): 7.2-7.7; 11.7-12.2; 16.2-16.7; 16.7-17.2; 19.6-20.1; 19.8-20.3; 22.2-22.7, and 22.9-23.4.

### CROSS-REFERENCE TO RELATED APPLICATIONS

This application is a Section 371 of International Application No. PCT/EP2012/003691, filed Sep. 4, 2012, which was published in the German language on Mar. 28, 2013, under International Publication No. WO 2013/041182 A1 and the disclosure of which is incorporated herein by reference.

### BACKGROUND OF THE INVENTION

The present invention relates to crystalline idarubicin hydrochloride, a method for its production, and a pharmaceutical composition containing this crystalline idarubicin hydrochloride.

Idarubicin (4-demethoxydaunomycin; (1S,3S)-3-acetyl-3,5,12-trihydroxy-6,11-dioxo-1,2,3,4,6,11-hexahydrotetracene-1-yl 3-amino-2,3,6-trideoxo-α-L-/yxo-hexopyranoside) and its acid addition salts, such as idarubicin hydrochloride, are compounds from the group of anthracyclines, which have been used since the 1980s as cytostatics for the treatment of various types of tumors.

A method for producing idarubicin hydrochloride emerges from U.S. Pat. No. 4,046,878. In this method, 4-demethoxydaunomycinone is condensed with 1-chlorine-2,3,6-trideoxy-3-trifluoroacetamido-4-trifluoroacetoxy-α-L-lyxopyranose in the presence of a mercury halide. The condensation product is initially converted with methanol and then with sodium hydroxide and afterwards transformed into the acid addition salt with hydrochloric acid.

Another method for producing idarubicin hydrochloride, which is based on the glycosylation of idarubicinonaglycone, was described by J. Swenton, Tetrahedron 40:4625 (1984).

It is known that many active pharmaceutical ingredients, when they are present in amorphous form or as a mixture of several different crystalline modifications, are not adequately stable, are of poor solubility, and can be processed only with difficulty. Therefore, it is desirable to provide active pharmaceutical ingredients in a stable crystalline modification.

For the idarubicin hydrochloride known from prior art, it has been found that it does not exist in a stable crystalline modification. Accordingly, in the currently available idarubicin hydrochloride, a steady decomposition has been observed during storage under typical storage conditions. This decomposition is traced back to the hydrolysis of the sugar group of the idarubicin hydrochloride, leading to a corresponding increase in 4-demethoxydaunomycinone. A highly contaminated idarubicin hydrochloride, however, is not acceptable for pharmaceutical compositions.

A crystalline modification of idarubicin hydrochloride is disclosed in Polish Patent PL 195 417 B1. This crystalline modification of idarubicin hydrochloride is produced by crystallizing idarubicin hydrochloride from a mixture of methanol and isopropanol, washing the resulting crystals with isopropanol, and then again crystallizing idarubicin hydrochloride from a mixture of water and isopropanol.

In PL 195 417 B1 the hypothesis is stated that idarubicin hydrochloride occurs in different modifications and therefore is polymorphous.

In consideration of the cited prior art, it would be desirable to have available, in addition to the crystalline modification known from PL 195 417 B1, another crystalline modification of idarubicin hydrochloride. This modification should preferably exhibit improved stability compared with the known idarubicin hydrochloride at various storage conditions, in particular various temperatures.

### BRIEF SUMMARY OF THE INVENTION

The invention is therefore based on the object of providing an alternative crystalline form of idarubicin hydrochloride. This crystalline form of idarubicin hydrochloride should additionally exhibit a high stability, so that it is especially suitable for use as an active pharmaceutical ingredient.

Furthermore, the object of the invention is to provide a method for producing such a crystalline idarubicin hydrochloride and also a pharmaceutical composition containing such a crystalline idarubicin hydrochloride.

The invention consequently provides a method for producing crystalline idarubicin hydrochloride, including the following steps:

(i) Production of a mixture containing (a) idarubicin hydrochloride, (b) at least one alcohol selected from the group consisting of 1-butanol, 2-butanol, and 1-pentanol, and (c) water; and

(ii) Crystallization of idarubicin hydrochloride from this mixture.

Furthermore, crystalline idarubicin hydrochloride is provided that has an x-ray diffraction pattern in which reflexes occur at least at diffraction angles in the following ranges (in 2Θ) (at least one reflex for each specified range): 7.2-7.7; 11.7-12.2; 16.2-16.7; 16.7-17.2; 19.6-20.1; 19.8-20.3; 22.2-22.7, and 22.9-23.4.

The invention also provides a pharmaceutical composition, which contains the crystalline idarubicin hydrochloride described above as well as a pharmaceutically acceptable carrier.

Crystalline idarubicin hydrochloride is produced according to the invention. This crystalline idarubicin hydrochloride is characterized at least by a powder x-ray diffraction pattern in which reflexes occur at least at diffraction angles in the following ranges (in 2Θ) (at least one reflex for each specified range): 7.2-7.7; 11.7-12.2; 16.2-16.7; 16.7-17.2; 19.6-20.1; 19.8-20.3; 22.2-22.7, and 22.9-23.4.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a typical powder x-ray diffraction diagram of the crystalline idarubicin hydrochloride produced according to an embodiment of the invention;

FIG. 2 is a typical DSC diagram of the crystalline idarubicin hydrochloride according to an embodiment of the invention; and

FIG. 3 is a graph of the results of the stability tests of Example 4 plotting percentage of idarubicin versus storage time in weeks.

### DETAILED DESCRIPTION OF THE INVENTION

According to one preferred embodiment, the crystalline idarubicin hydrochloride has a powder x-ray diffraction pattern in which reflexes occur at least at the following diffraction angles 2Θ: 7.54; 12.06; 16.52; 16.93; 19.86; 20.14; 22.47; 23.13.

According to one especially preferred embodiment, the crystalline idarubicin hydrochloride is characterized by a powder x-ray diffraction pattern in which reflexes having relative intensities P(%) occur at least at diffraction angles (2Θ) according to the following table:

| | | Preferred relative |
|---|---|---|
| Diffraction angle (2Θ) | Relative intensity P (%) | intensity P (%) |
| 7.54 | 16 | 12-20 |
| 12.06 | 15 | 12-20 |
| 16.52 | 30 | 25-35 |
| 16.93 | 22 | 18-26 |
| 19.86 | 32 | 27-37 |
| 20.14 | 51 | 44-58 |
| 22.47 | 100 | 100 |
| 23.13 | 18 | 14-22 |

According to another preferred embodiment, the crystalline idarubicin hydrochloride according to the invention is characterized by a powder x-ray diffraction pattern in which reflexes occur at least at the diffraction angles in the following ranges (in 2Θ) (at least one reflex for each specified range): 5.2-5.7; 7.2-7.7; 7.7-8.2; 11.7-12.2; 16.2-16.7; 16.7-17.2; 18.8-19.3; 19.6-20.1; 19.8-20.3; 22.2-22.7; 22.9-23.4; 23.3-23.8; 24.8-25.3; 26.8-27.3; 27.3-27.8; and 32.3-32.8.

According to yet another preferred embodiment, this crystalline idarubicin hydrochloride is preferably characterized by an x-ray diffraction pattern in which reflexes occur at least at the following diffraction angles 2Θ: 5.36; 7.54; 7.66; 12.06; 16.52; 16.93; 19.12; 19.86; 20.14; 22.47; 23.13; 23.54; 24.96; 26.97; 27.66, and 32.64.

According to another especially preferred embodiment, the crystalline idarubicin hydrochloride is characterized by a powder x-ray diffraction pattern in which reflexes having relative intensities P(%) occur at least at diffraction angles (2Θ) according to the following table:

| | | Preferred relative |
|---|---|---|
| Diffraction angle (2Θ) | Relative intensity P (%) | intensity P (%) |
| 5.36 | 2-6 | 4 |
| 7.54 | 12-20 | 16 |
| 7.66 | 10-18 | 14 |
| 12.06 | 12-20 | 15 |
| 16.52 | 25-35 | 30 |
| 16.93 | 18-26 | 22 |
| 19.12 | 5-15 | 10 |
| 19.86 | 27-37 | 32 |
| 20.14 | 44-58 | 51 |
| 22.47 | 100 | 100 |
| 23.13 | 14-22 | 18 |
| 23.54 | 4-14 | 9 |
| 24.96 | 3-13 | 8 |
| 26.97 | 8-18 | 13 |
| 27.66 | 2-10 | 6 |
| 32.64 | 2-12 | 7 |

According to the invention it can be preferred that the term “reflex” is understood to be the signal of respective peaks in the x-ray diffraction diagram having the maximum intensity.

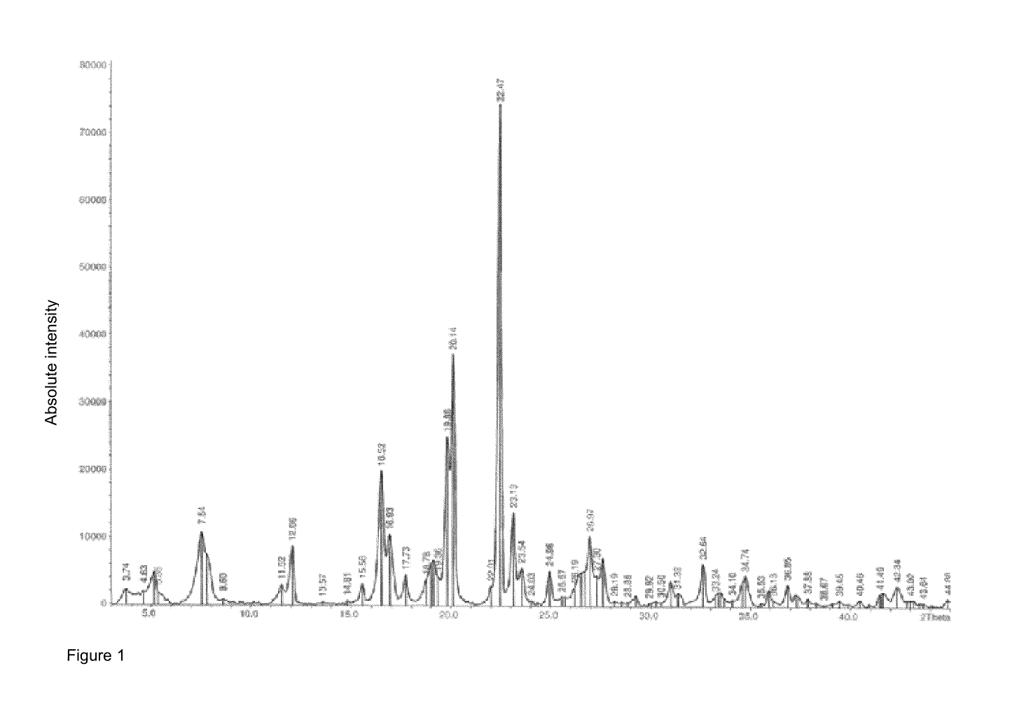

A typical powder x-ray diffraction diagram of the crystalline idarubicin hydrochloride produced according to an embodiment of the invention is shown in FIG. 1.

The above values are obtained preferably from x-ray diffraction measurements conducted with a powder x-ray diffractometer from the company Stoe (Darmstadt) by an IPPSD detector (image-plate position-sensitive detector) using Cu-Kα radiation (λ=1.5406 Å) (Ge monochromator). The measurement range for 2Θ is 3 to 79. The measurement instruments are calibrated against Si 5N=99.999%.

The crystalline idarubicin hydrochloride according to the invention preferably has a peak in a Differential Scanning calorimetry (DSC) diagram having a maximum intensity in the temperature range of 180-205° C., more preferably having a maximum intensity in the temperature range of 185-200° C., and even more preferably having a maximum intensity in the temperature range of 190-200° C. This peak is preferably an exothermic peak.

The Differential Scanning calorimetry (DSC) diagram can be obtained within the framework of the invention, for example, by heating a sample of the crystalline idarubicin hydrochloride (for example corresponding to a quantity of 1-8 mg idarubicin hydrochloride) to 30-350° C. at a heating rate of 10-20 K/min, preferably at a heating rate of 10 K/min, in a DSC calorimeter.

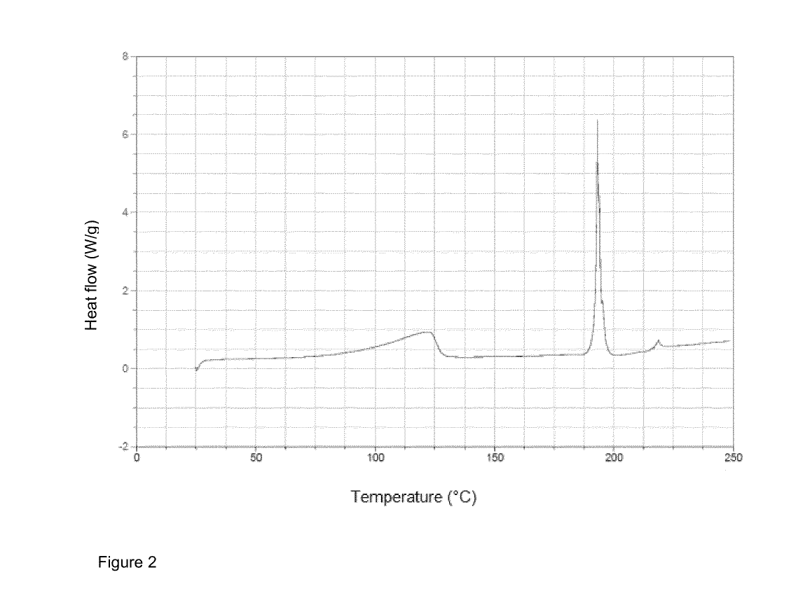

A typical DSC diagram of the crystalline idarubicin hydrochloride according to an embodiment of the invention is shown in FIG. 2.

The idarubicin hydrochloride according to the invention preferably has a purity of at least 95%, more preferably a purity of at least 99%, even more preferably a purity of at least 99.5%, especially preferred a purity of at least 99.8%, most especially preferred a purity of at least 99.9%, and in particular a purity of at least 99.99%.

For producing crystalline idarubicin hydrochloride, in step (i) first a mixture is prepared containing (a) idarubicin hydrochloride, (b) at least one alcohol selected from the group consisting of 1-butanol, 2-butanol, and 1-pentanol, and (c) water.

The mixture of step (i) can be, for example, a solution or a suspension.

The mixture of step (i) contains idarubicin hydrochloride.

The idarubicin hydrochloride can be produced in a known manner, for example using a fermentative process, a chemical synthesis process, or a mixture thereof (for example semi-synthetic process).

According to one possible embodiment of the invention, idarubicin hydrochloride is produced in situ in the mixture of step (i), in which idarubicin free base is converted into idarubicin hydrochloride. This conversion can take place, for example, by adding hydrogen chloride. Hydrogen chloride can be added, for example, as hydrochloric acid. Furthermore, it is also possible for this purpose to use a hydrogen chloride-containing solution, for example an alcoholic solution containing hydrogen chloride.

According to the invention it can be advantageous that the content of idarubicin hydrochloride is at least 3 g/l and more preferably at least 5 g/l, relative to the total volume of the mixture in step (i). The content of idarubicin hydrochloride can be preferably up to 100 g/l, more preferably up to 50 g/l, even more preferably up to 30 g/l, and especially preferred up to 20 g/l, relative to the total volume of the mixture in step (i). Preferably, the content of idarubicin hydrochloride lies in the range of 3-100 g/l, more preferably in the range of 3-50 g/l, even more preferably in the range of 3-30 g/l, and especially preferred in the range of 3-20 g/l, relative to the total volume of the mixture in step (i). A concentration of idarubicin hydrochloride in this range leads to a surprisingly high yield of crystalline idarubicin hydrochloride.

The mixture of step (i) further contains at least one alcohol selected from the group consisting of 1-butanol, 2-butanol, and 1-pentanol. This alcohol is preferably 1-butanol.

The presence of an alcohol selected from the group consisting of 1-butanol, 2-butanol, and 1-pentanol, in particular 1-butanol, surprisingly contributes to preventing the formation of gel that is otherwise typical for idarubicin hydrochloride and that is an obstacle to the crystallization of idarubicin hydrochloride. Accordingly, the presence of at least one alcohol selected from the group consisting of 1-butanol, 2-butanol, and 1-pentanol promotes, to a special degree, the growth of idarubicin hydrochloride crystals.

According to one preferred embodiment, the content of the at least one alcohol (b) selected from the group consisting of 1-butanol, 2-butanol, and 1-pentanol is at least 7 volume percent and more preferably at least 10 volume percent, relative to the total volume of the mixture of step (i). The content of the at least one alcohol (b) selected from the group consisting of 1-butanol, 2-butanol, and 1-pentanol is preferably up to 96 volume percent, more preferably up to 92 volume percent and even more preferably up to 80 volume percent, relative to the total volume of the mixture of step (i). The content of the at least one alcohol (b) selected from the group consisting of 1-butanol, 2-butanol, and 1-pentanol accordingly preferably lies in the range of 7-96 volume percent, more preferably in the range of 10-96 volume percent, and even more preferably in the range of 10-92 volume percent, relative to the total volume of the mixture of step (i). At a concentration of less than 7 volume percent of the at least one alcohol selected from the group consisting of 1-butanol, 2-butanol, and 1-pentanol, relative to the total volume of the mixture, it has been shown that the tendency for crystallization of idarubicin hydrochloride decreases considerably.

The mixture in step (i) also contains water.

According to one preferred embodiment of the invention, the content of water is at least 4.0 volume percent, more preferably at least 4.1 volume percent, even more preferably at least 4.2 volume percent, especially preferred at least 4.3 volume percent, most especially preferred at least 4.4 volume percent, and in particular at least 5 volume percent, relative to the total volume of the mixture of step (i). The content of water can here equal preferably up to 12.0 volume percent, more preferably up to 10.0 volume percent, and even more preferably up to 8.0 volume percent, relative to the total volume of the mixture of step (i). The content of water can therefore preferably lie in the range of 4.0-12.0 volume percent, more preferably in the range of 4.0-10.0 volume percent, and even more preferably in the range of 4.0-8.0 volume percent, relative to the total volume of the mixture of step (i).

According to another preferred embodiment, the mixture of step (i) contains at least one additional alcohol (d). This additional alcohol (d) is preferably selected from the group consisting of methanol, ethanol, 1-propanol, and 2-propanol. The content of the at least one additional alcohol (d), if contained in the mixture from step (i), is preferably at least 0.1 volume percent, more preferably at least 1.0 volume percent, and even more preferably at least 5.0 volume percent, relative to the volume of the mixture of step (i). The content of the additional alcohol (d) preferably equals up to 86.0 volume percent, more preferably up to 80.0 volume percent, even more preferably up to 65.0 volume percent, especially preferred up to 50.0 volume percent, and most especially preferred up to 40.0 volume percent, relative to the volume of the mixture of step (i). The content of the at least one alcohol (d) therefore preferably lies in the range of 0-86.0 volume percent, more preferably in the range of 0.1-86.0 volume percent, even more preferably in the range of 1.0-80.0 volume percent, especially preferred in the range of 5.0-65.0 volume percent, very especially preferred in the range of 5.0-50.0 volume percent, and in particular in the range of 5.0-40.0 volume percent, relative to the volume of the mixture of step (i).

If an additional alcohol (d) is contained in the mixture, then it can be preferred that the ratio of the volume of this at least one additional alcohol (d) to the volume of the at least one alcohol (b) selected from the group consisting of 1-butanol, 2-butanol, and 1-pentanol is at most 2:1, more preferably at most 1:1, even more preferably at most 1:2, especially preferred at most 1:3, and most especially preferred at most 1:4. In addition, it can be preferred that the ratio of the volume of this at least one additional alcohol (d) to the volume of the at least one alcohol (b) selected from the group consisting of 1-butanol, 2-butanol, and 1-pentanol lies in the range of 1:1 to 1:20, more preferably in the range of 1:1 to 1:10, and even more preferably in the range of 1:1 to 1:7.

According to yet another preferred embodiment, the mixture of step (i) contains at least one halogenated hydrocarbon compound (e). This at least one halogenated hydrocarbon compound (e) is preferably at least one chlorinated hydrocarbon compound. The halogenated hydrocarbon compound (e) is here preferably selected from the group consisting of dichloromethane and trichloromethane. The content of the at least one halogenated hydrocarbon compound (e), if contained in the mixture of step (i), is preferably at least 0.1 volume percent, relative to the volume of the mixture of step (i). Preferably, the content of the at least one halogenated hydrocarbon compound (e) equals up to 86.0 volume percent, more preferably up to 60.0 volume percent, and even more preferably up to 40.0 volume percent, relative to the volume of the mixture of step (i). The content of the at least one halogenated hydrocarbon compound (e) accordingly preferably lies in the range of 0-86 volume percent, more preferably in the range of 0.1-86.0 volume percent, even more preferably in the range of 0.1-60.0 volume percent, and especially preferred in the range of 0.1-40.0 volume percent, relative to the volume of the mixture of step (i).

A mixture that has proven especially advantageous in step (i) has the following composition: (a) idarubicin hydrochloride, (b) 10-96 volume percent of at least one alcohol selected from the group consisting of 1-butanol, 2-butanol, and 1-pentanol, (c) 4.0-8.0 volume percent water, (d) 0-86 volume percent of at least one additional alcohol selected from the group consisting of methanol, ethanol, 1-propanol, and 2-propanol, and (e) 0-86 volume percent of at least one halogenated hydrocarbon compound, relative to the total volume of the mixture of step (i).

A pH value of the mixture from step (i) in the range of 2.5-4.5 has proven especially advantageous for the crystallization. An optimum crystallization is here obtained if the pH value of the mixture from step (i) lies in the range of 2.8-4.5, more preferably in the range of 3.0-4.5, and in particular in the range of 3.0-4.0. If the mixture is produced by adding the at least one alcohol (b) and water (c) to idarubicin hydrochloride as a solid, then the mixture typically already has a pH value in this range. If the production of the mixture takes place by adding the at least one alcohol (b) to a solution containing idarubicin hydrochloride, then the mixture could have a higher pH value. In this case, the pH value can be adjusted to the preferred range, for example by adding hydrogen halide.

The mixture of step (i) can be produced in a technically conventional way.

For producing the mixture, for example, idarubicin hydrochloride in already dissolved form or as a solid can be used. If idarubicin hydrochloride in a dissolved form is introduced into the mixture, this solution can contain one or more solvents. The at least one solvent is preferably selected from the group consisting of water, alcohols, and halogenated hydrocarbon compounds. As alcohols, methanol, ethanol, 1-propanol, 2-propanol, and mixtures thereof can be preferred. As the halogenated hydrocarbon compound, chloroform and dichloromethane can be preferred. According to one preferred embodiment, the pH value of the solution containing idarubicin hydrochloride lies in the range of 2.5 to 4.5 and more preferably in the range of 3 to 4.

If idarubicin hydrochloride is used as a solid, then it can be amorphous idarubicin hydrochloride, crystalline idarubicin hydrochloride, mixtures of various crystalline forms of idarubicin hydrochloride, or mixtures thereof.

Furthermore, it is possible to use idarubicin base for producing the mixture of step (i) and to produce idarubicin hydrochloride from this in situ. The production of idarubicin hydrochloride from idarubicin base in situ can be performed, for example, by adding hydrochloric acid or a hydrogen halide-containing solution, for example a hydrogen halide-containing isopropanolic solution, to a solution or a suspension of idarubicin base.

The mixture of step (i) can be produced, for example, by combining idarubicin hydrochloride (for example as a solid, in suspension, or in solution), at least one alcohol selected from the group consisting of 1-butanol, 2-butanol, and 1-pentanol, and water, wherein the content of water is at least 4.0 volume percent, relative to the total volume of the mixture.

The mixture of step (i) can likewise be produced, for example, by combining idarubicin base, at least one alcohol selected from the group consisting of 1-butanol, 2-butanol, and 1-pentanol, and water, and in this mixture idarubicin hydrochloride is formed in situ by adding hydrogen chloride. Hydrogen chloride can here be added to the mixture, for example, as hydrochloric acid or in an alcoholic solution (such as an isopropanolic solution).

For producing crystalline idarubicin hydrochloride, in a step (ii) idarubicin hydrochloride is crystallized out of the mixture from step (i).

The crystallization of idarubicin hydrochloride can be triggered in a simple manner:

According to a first preferred, superior embodiment of the invention, the crystallization of idarubicin hydrochloride is caused by reduction of the water content in the mixture from step (i).

Preferably, according to this embodiment, the content of water in the mixture of step (i) is at least 4.0 volume percent, relative to the total volume of the mixture of step (i). The crystallization of idarubicin hydrochloride is triggered in this case by reducing the content of water in the mixture from step (i) to less than 4.0 volume percent, relative to the total volume of the mixture of step (i). Preferably, the content of water in the mixture of step (i) is reduced to less than 3.9 volume percent, more preferably to less than 3.8 volume percent, even more preferably to less than 3.7 volume percent, especially preferred to less than 3.5 volume percent, most especially preferred to less than 3.2 volume percent, and in particular to less than 3.0 volume percent, relative to the total volume of the mixture of step (i).

This reduction of the water content of the mixture from step (i) to less than 4.0 volume percent, relative to the total volume of the mixture of step (i), can be conducted in various ways.

According to one preferred embodiment, the content of water in the mixture of step (i) is reduced by distillation.

The distillation can take place, for example, at a reduced pressure. Preferably, the distillation is conducted at a pressure in the range of 10-800 mbar, more preferably at a pressure in the range of 20-600 mbar, even more preferably at a pressure in the range of 30-400 mbar, especially preferred at a pressure in the range of 40-300 mbar, and most especially preferred at a pressure in the range of 50-200 mbar.

The distillation is typically conducted at a temperature of more than 25° C. Preferably, the distillation takes place at a temperature in the range of 30-90° C., more preferably at a temperature in the range of 40-80° C., even more preferably at a temperature in the range of 40-70° C., and especially preferred at a temperature in the range of 60-70° C.

If the mixture from step (i) was heated for reducing the water content to less than 4.0 volume percent, then the resulting mixture, which is preferably present as a suspension, is preferably cooled. The cooling of the obtained mixture can take place, for example, in stages. For example, it can be expedient to cool the resulting mixture stepwise to temperatures in the range of 68-72° C., 63-67° C., 58-62° C., 53-57° C., and 20-28° C., more preferably to temperatures of 70° C., 65° C., 60° C., 55° C., and 22° C., wherein each temperature is held for a certain time period that is preferably 1-120 minutes, more preferably 2-60 minutes, and even more preferably 5-30 minutes.

It has been proven advantageous to perform a reduction of the water content in the mixture from step (i) by a reduction of the total volume of the mixture from step (i) to 50-95%, more preferably to 50-90%, even more preferably to 60-90%, especially preferred to 65-90%, most especially preferred to 70-90%, and in particular to 75-85%, relative to the total volume of the mixture of step (i).

It has been shown that just the reduction of the content of water in the mixture from step (i) to less than 4.0 volume percent, relative to the total volume of the mixture of step (i), for example during distillation, easily leads to a crystallization of idarubicin hydrochloride in a high yield.

Furthermore, it has been shown that the crystalline idarubicin hydrochloride of the present invention surprisingly has an extraordinarily high thermodynamic stability. In particular, the crystalline idarubicin hydrochloride of the present invention is thermodynamically more stable than amorphous idarubicin hydrochloride. Therefore, for crystallization of idarubicin hydrochloride from a solution, the crystalline idarubicin hydrochloride according to the invention is therefore typically obtained directly.

According to an alternative, superior embodiment of the invention, the crystallization of idarubicin hydrochloride is triggered by letting the mixture from step (i) stand.

According to this embodiment it can be advantageous if the mixture of step (i) is a suspension.

Surprisingly, it was found that the crystallization of idarubicin hydrochloride was produced just by letting the mixture from step (i) stand.

Here, the mixture from step (i) is preferably stirred.

It is also possible to heat the mixture from step (i) for the crystallization of idarubicin hydrochloride. Preferably, the mixture from step (i) is heated to a temperature of at least 25° C., more preferably to a temperature of at least 30° C., even more preferably to a temperature of at least 40° C., especially preferred to a temperature of at least 50° C., most especially preferred to a temperature of at least 60° C., and in particular to a temperature of at least 65° C. The heating of mixture from step (i) preferably takes place at a temperature of at most 95° C., more preferably at a temperature of at most 90° C., even more preferably at a temperature of at most 85° C., especially preferred at a temperature of at most 80° C., and most especially preferred at a temperature of at most 75° C. Accordingly, the mixture from step (i) is preferably heated to a temperature in the range of 25° C.-95° C., more preferably to a temperature in the range of 30° C.-90° C., even more preferably to a temperature in the range of 40° C.-85° C., especially preferred to a temperature in the range of 50° C.-80° C., and most especially preferred to a temperature in the range of 60° C.-75° C.

The heating of the mixture from step (i) preferably takes place for a time period of at least 10 minutes, more preferably for a time period of at least 30 minutes, even more preferably for a time period of at least 60 minutes, especially preferred for a time period of at least 2 hours, and most especially preferred for a time period of at least 3 hours. The heating of the mixture from step (i) preferably takes place for a time period of at most 24 hours, more preferably for a time period of at most 12 hours, even more preferably for a time period of at most 10 hours, especially preferred for a time period of at most 8 hours, and most especially preferred for a time period of at most 7 hours. Accordingly, the heating of the mixture from step (i) preferably takes place for a time period in the range of 10 minutes-48 hours, more preferably for a time period in the range of 30 minutes-12 hours, even more preferably for a time period in the range of 60 minutes-10 hours, especially preferred for a time period in the range of 2-8 hours, most especially preferred for a time period in the range of 3-7 hours, and in particular for a time period in the range of 4-6 hours.

It can be advantageous to then let the obtained mixture cool. The cooling can take place, for example, at a temperature that lies preferably at least 5° C., more preferably at least 10° C., even more preferably at least 20° C., especially preferred at least 30° C., most especially preferred at least 40° C., and in particular at least 50° C. below the temperature to which the mixture from step (i) had previously been heated. Accordingly, the cooling of the obtained mixture can preferably take place at a temperature in the range of 5-40° C., more preferably at a temperature in the range of 10° C.-30° C., and even more preferably at a temperature in the range of 15° C.-25° C.

After cooling, the obtained mixture can optionally be further stirred. Stirring can take place preferably for at least an additional 10 minutes, more preferably for at least an additional 60 minutes, even more preferably for at least an additional 2 hours, especially preferred for at least an additional 4 hours, most especially preferred for at least an additional 8 hours, and in particular for at least an additional 12 hours.

According to this superior embodiment of the invention, the idarubicin hydrochloride contained as a solid in the suspension is gradually converted into the thermodynamically more stable crystalline idarubicin hydrochloride of the present invention.

The isolation of the crystalline idarubicin hydrochlorides from the mixture from step (ii) can take place in a technically conventional manner.

According to one preferred embodiment, the crystals of idarubicin hydrochloride are isolated from the mixture from step (ii) by filtration.

The crystalline idarubicin hydrochloride obtained after the isolation from the mixture from step (ii) can be washed, if necessary. The washing can be conducted with a solvent suitable for this purpose, in which idarubicin hydrochloride preferably has a lower solubility than in at least one of the compounds (b) and (c) contained in the mixture from step (i). Ketones, such as acetone, as well as ethers, such as tert-butyl methyl ether, have proven to be especially suitable solvents for the washing of crystalline idarubicin hydrochloride.

The crystalline idarubicin hydrochloride isolated from the rest of the mixture from step (ii) and optionally washed can then be dried. The drying can be performed, for example, at a reduced pressure.

The crystalline idarubicin hydrochloride obtained according to the invention can be used for producing a pharmaceutical composition.

This pharmaceutical composition can preferably be provided for oral administration, for enteral administration, or for parenteral administration. Consequently, the pharmaceutical composition is preferably provided in the form of tablets (for example coated or uncoated tablets), capsules, solutions, suspensions, or lyophilizates for reconstitution before an injection.

This pharmaceutical composition can preferably have a fluid or solid consistency at a temperature of 25° C. and a pressure of 1.013 bar.

According to one preferred embodiment, the pharmaceutical composition contains the crystalline idarubicin hydrochloride according to the invention as a solid.

The pharmaceutical composition contains, in addition to the crystalline idarubicin hydrochloride according to the invention as a solid, also a pharmaceutically acceptable carrier. As the pharmaceutically acceptable carrier, pharmaceutically acceptable carriers typically used for pharmaceutical compositions can be used. The choice of the pharmaceutically acceptable carrier is, in a known way, dependent on, among other things, the dosage form of the pharmaceutical composition. Suitable pharmaceutically acceptable carriers are therefore, for example, polypeptides (for example gelatins), polysaccharides (for example cellulose, dextran, or dextrin), disaccharides (for example lactose), alginates (for example sodium alginate), water, and mixtures thereof. For the pharmaceutical composition of the present invention, polypeptides (for example gelatins), polysaccharides (for example cellulose, dextran, or dextrin), alginates (for example sodium alginate), and mixtures thereof are preferably used as the carrier.

The pharmaceutical composition can include, in addition to the crystalline idarubicin hydrochloride and the pharmaceutically acceptable carrier, additional substances that are preferably harmless and compatible with regard to the crystalline idarubicin hydrochloride. These additional substances include, in particular, emulsifiers, excipients, and additives. As excipients, for example, fillers (for example monoglycerides, diglycerides, triglycerides, and mixtures thereof), extenders, binding agents (for example polyvinyl alcohol, polyvinylpyrrolidone, gum arabic, mannitol, sorbitol, glycerin, and mixtures thereof), stabilizing agents, coloring agents (for example iron-III-oxide, titanium dioxide, and mixtures thereof), buffering agents, flavoring agents, and odorous substances can be used.

The production of the pharmaceutical composition can take place in a technically conventional way. For example, the pharmaceutically acceptable carrier can be mixed or filled with the crystalline idarubicin hydrochloride in a suitable concentration, or the crystalline idarubicin hydrochloride is dissolved in the pharmaceutically acceptable carrier.

The invention will be described below using examples that do not, however, limit the scope of protection.

### EXAMPLES

### Example 1

1 g idarubicin hydrochloride was dissolved in a mixture of 8 ml water and 92 ml 1-butanol. Here, the mixture was heated to 80° C., in order to completely dissolve the solids. 20 ml of this mixture was slowly removed by distillation in a vacuum, in order to reduce the water content to less than 4.0 volume percent, relative to the total volume of the mixture. A suspension was thereby formed, which was cooled to 20° C. within 6 hours. The suspension was stirred for an additional 12 hours at this temperature. The crystals contained in the suspension as solids were filtered and washed with 20 ml acetone. The crystals were then dried for 12 hours under vacuum. A yield of idarubicin hydrochloride of 92% resulted.

### Example 2

1 g idarubicin free base was introduced into 100 ml of a mixture of 80 ml chloroform and 20 ml methanol. The pH value of this mixture was then set to a value in the range of 3.5-4.0 by adding 0.1 M isopropanolic HCl solution. This mixture was mixed with 100 ml 1-butanol 10 ml water. Then, the chloroform was slowly removed from the mixture by distillation at 60° C. Thereafter, 20 ml of this mixture was slowly removed by distillation in a vacuum at 80° C., in order to reduce the water content to less than 4.0 volume percent, relative to the total volume of the mixture. Here, a suspension formed that was cooled to 20° C. within 6 hours. At this temperature, the suspension was stirred for an additional 12 hours. The crystals contained in the suspension as solids were filtered and washed with 20 ml acetone. The crystals were then dried for 12 hours under vacuum. A yield of idarubicin hydrochloride of 95% resulted.

### Example 3

A suspension was produced from 1 g amorphous idarubicin hydrochloride in 80 ml 1-butanol and 4 ml water. This suspension was heated to a temperature of 70° C. and stirred at this temperature for 4-6 hours. The suspension was then slowly cooled to 20° C. and stirred for an additional 12 hours. The crystals obtained were filtered and washed briefly with 20 ml acetone. Thereafter, the crystals were dried for 12 hours under vacuum. A yield of idarubicin hydrochloride of 95% resulted.

### Example 4

The crystalline idarubicin hydrochloride obtained in Example 1 was studied for its stability relative to storage at temperatures of 25° C. and 40° C. for various time periods. For this purpose, aliquots of the obtained crystalline idarubicin hydrochlorides were encapsulated individually. One half of the aliquots were stored in a drying chamber at 25° C., the other half were stored in a different drying chamber at 40° C. After the times specified below, individual samples were taken from the drying chambers and the content of idarubicin hydrochloride was analyzed by high performance liquid chromatography (HPLC).

The results of the test for stability at a storage temperature of 25° C. are set forth in the following table.

| Storage period in weeks at | |
|---|---|
| 25° C. | Purity of idarubicin hydrochloride |
| 0 | 99.95 |
| 1 | 99.90 |
| 2 | 99.87 |
| 4 | 99.93 |
| 8 | 99.84 |
| 24 | 99.91 |

The results of the test for stability at a storage temperature of 40° C. are set forth in the following table.

| Storage period in weeks at | |
|---|---|
| 40° C. | Purity of idarubicin hydrochloride |
| 0 | 99.95 |
| 1 | 99.87 |
| 2 | 99.87 |
| 4 | 99.92 |
| 8 | 99.83 |
| 24 | 99.91 |

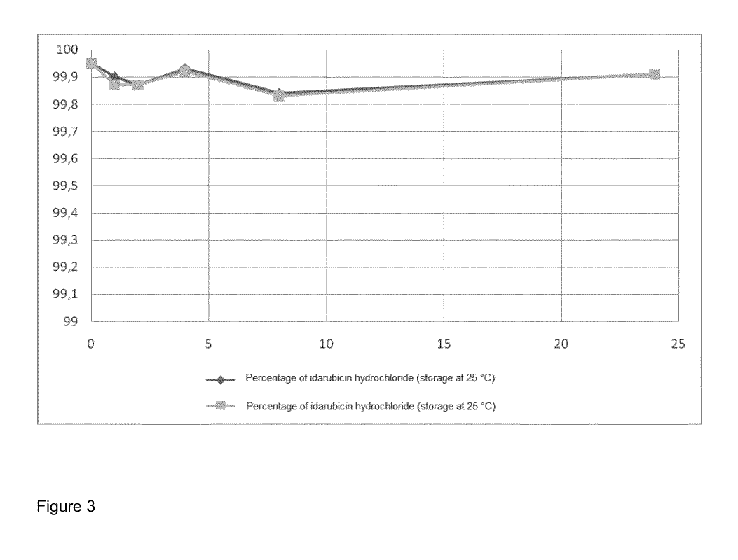

The results of the stability tests are compiled in FIG. 3.

It has been shown that the crystalline idarubicin hydrochloride according to the invention exhibits an extraordinarily high stability for long storage under elevated temperatures.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1-16. (canceled)

17. A method for production of crystalline idarubicin hydrochloride, the method comprising steps of:
(i) producing a mixture containing (a) idarubicin hydrochloride, (b) a least one alcohol selected from 1-butanol, 2-butanol, and 1-pentanol, and (c) water; and
(ii) crystallizing idarubicin hydrochloride from this mixture.

18. The method according to claim 17, wherein the at least one alcohol (b) is 1-butanol.

19. The method according to claim 17, wherein in step (i) the idarubicin hydrochloride is present in a range of 3-100 g/l, relative to a total volume of the mixture of step (i).

20. The method according to claim 17, wherein in step (i) the at least one alcohol (b) is present in a range of 10-96 volume percent, relative to a total volume of the mixture of step (i).

21. The method according to claim 17, wherein the water is present in an amount of at least 4.0 volume percent, relative to a total volume of the mixture of step (i).

22. The method according to claim 17, wherein in step (i) the water (c) is present in a range of 4.0-8.0 volume percent, relative to a total volume of the mixture of step (i).

23. The method according to claim 17, wherein the mixture of step (i) contains at least one additional alcohol (d) selected from methanol, ethanol, 1-propanol, and 2-propanol.

24. The method according to claim 17, wherein the mixture of step (i) further contains a halogenated hydrocarbon compound (e).

25. The method according to claim 24, wherein the halogenated hydrocarbon compound (e) is selected from dichloromethane and trichloromethane.

26. The method according to claim 17, wherein the mixture of step (i) has a pH in a range of 2.5-4.5.

27. The method according to claim 17, wherein the crystalline idarubicin hydrochloride is separated from the rest of the mixture.

28. The method according to claim 17, wherein for the crystallizing of idarubicin hydrochloride at least one of the following steps is performed:
(ii-1) allowing the mixture from step (i) to stand; and
(ii-2) reducing water content in the mixture from step (i) to less than 4.0 volume percent relative to a total volume of the mixture, while retaining crystalline idarubicin hydrochloride.

29. The method according to claim 28, wherein the reducing of the water content in the mixture from step (i) takes place by distillation.

30. The method according to claim 29, wherein the distillation takes place at a temperature in a range of 60-80° C. under reduced pressure.

31. The method according to claim 30, wherein the reduced pressure is a pressure of 50-200 mbar.

32. Crystalline idarubicin hydrochloride, characterized by a powder x-ray diffraction pattern in which at least reflexes at diffraction angles occur in the following ranges (in 2Θ): 7.2-7.7; 11.7-12.2; 16.2-16.7; 16.7-17.2; 19.6-20.1; 19.8-20.3; 22.2-22.7, and 22.9-23.4.

33. Crystalline idarubicin hydrochloride according to claim 32, characterized by a peak in a Differential Scanning calorimetry (DSC) diagram having a maximum intensity in a temperature range of 180-205° C.

34. A pharmaceutical composition containing crystalline idarubicin hydrochloride according to claim 32 as a solid in a pharmaceutically acceptable carrier.

35. A pharmaceutical composition containing crystalline idarubicin hydrochloride according to claim 33 as a solid in a pharmaceutically acceptable carrier.
